(19) United States
(12) Patent Application Publication
(10) Pub. No.: US 2006/0154335 A1
(43) Pub. Date: Jul. 13, 2006

(54) Novel members of the steroid/thyroid superfamily and uses therefor

(75) Inventors: Ronald M. Evans, La Jolla, CA (US); Barry M. Forman, Irvine, CA (US); Steven A. Kliewer, Cary, NC (US); Estelita S. Ong, San Diego, CA (US); Bruce Blumberg, Irvine, CA (US)

Correspondence Address: FOLEY & LARDNER LLP, P.O. BOX 80278, SAN DIEGO, CA, 92138-0278, US

(73) Assignee: The Salk Institute for Biological Studies

(21) Appl. No.: 11/235,557

(22) Filed: Sep. 26, 2005

Related U.S. Application Data
(62) Division of application No. 08/484,215, filed on Jun. 7, 1995, now abandoned.
Application No. 08/484,215 is a continuation-in-part of application No. 08/270,643, filed on Jul. 1, 1994, now abandoned.

(51) Int. Cl. C07K 14/705; C07H 21/04; C12P 21/06

(52) U.S. Cl. 435/69.1; 435/320.1; 435/325; 530/350; 536/23.5

(57) ABSTRACT

Novel peroxisome proliferators-activated receptor subunits designated PPARγ and PPARδ are described. Nucleic acid sequences encoding the receptor subunits, expression vectors containing such sequences and host cells transformed with such vectors are also disclosed, as are heterodimeric PPAR receptors comprising at least one of the invention subunits, and methods for the expression of such novel receptors, and various uses therefor.

### RELATED APPLICATIONS

This application is a divisional of U.S. application Ser. No. 08/484,215, filed Jun. 7, 1995, now pending, which is a continuation-in-part of U.S. application Ser. No. 08/270,643, filed Jul. 1, 1994, now abandoned, the entire contents of each of which are hereby incorporated by reference herein.

### FIELD OF INVENTION

The present invention relates to novel members of the steroid/thyroid superfamily of receptors, as well as uses therefor.

### BACKGROUND OF THE INVENTION

Peroxisome proliferators are a structurally diverse group of compounds which, when administered to rodents, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, Ann. Rev. Cell Biol. 1:489-530 (1985); Vamecq and Draye, Essays Biochem. 24:1115-225 (1989); and Nelali et al., Cancer Res. 48:5316-5324 (1988)). Chemicals included in this group are the fibrate class of hypolipidermic drugs, herbicides, and phthalate plasticizers (Reddy and Lalwani, Crit. Rev. Toxicol. 12:1-58 (1983)). Peroxisome proliferation can also be elicited by dietary or physiological factors such as a high-fat diet and cold acclimatization.

Insight into the mechanism whereby peroxisome proliferators exert their pleiotropic effects was provided by the identification of a member of the nuclear hormone receptor superfamily activated by these chemicals (Isseman and Green, Nature 347-645-650 (1990)). This receptor, termed peroxisome proliferator activated receptor alpha (PPARα), was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid ω-hydroxylase (Gottlicher et al., Proc. Natl. Acad. Sci. USA 89:4653-4657 (1992); Tugwood et al., EMBO J. 11:433-439 (1992); Bardot et al., Biochem. Biophys. Res. Comm. 192:37-45 (1993); Muerhoff et al., J. Biol. Chem. 267:19051-19053 (1992); and Marcus et al., Proc. Natl. Acad. Sci. USA 90(12):5723-5727 (1993). The foregoing references support a physiological role for PPARα in the regulation of lipid metabolism. PPARα activates transcription by binding to DNA sequence elements, termed peroxisome proliferator response elements (PPRE), as a heterodimer with the retinoid X receptor. The retinoid X receptor is activated by 9-cis retinoic acid (see Kliewer et al., Nature 358:771-774 (1992), Gearing et al., Proc. Natl. Acad. Sci. USA 90:1440-1444 (1993), Keller et al., Proc. Natl. Acad. Sci. USA 90:2160-2164 (1993), Heyman et al., Cell 68:397-406 (1992), and Levin et al., Nature 355:359-361 (1992)). Since the PPARα-RXR complex can be activated by peroxisome proliferators and/or 9-cis retinoic acid, the retinoid and fatty acid signaling pathways are seen to converge in modulating lipid metabolism.

### BRIEF DESCRIPTION OF THE INVENTION

In accordance with the present invention, there are provided isolated mammalian peroxisome proliferators-activated receptor subunit proteins of the γ and α subtypes, and functional fragments thereof. In addition, there are provided isolated nucleic acids encoding mammalian peroxisome proliferator-activated receptor subunit proteins, as well as fragments thereof. There are also provided vectors containing the above-described nucleic acids, as well as cells containing such nucleic acids and/or vectors.

The present invention also provides methods for the recombinant production of mammalian peroxisome proliferator-activated receptor proteins comprising at least one PPAR subunit protein of the γ and δ subtype, and functional fragments thereof, as well as methods to identify clones encoding the above-described receptor subunit proteins, and functional fragments thereof.

Also provided by the present invention are methods for screening compounds to determine those which bind to mammalian peroxisome proliferator-activated receptor proteins comprising at least one PPAR subunit protein of the γ or δ subtype, or functional fragments thereof, as well as bioassays for evaluating whether test compounds are agonists or antagonists for receptor proteins of the invention, or functional modified forms of said receptor protein(s).

### BRIEF DESCRIPTION OF THE FIGS.

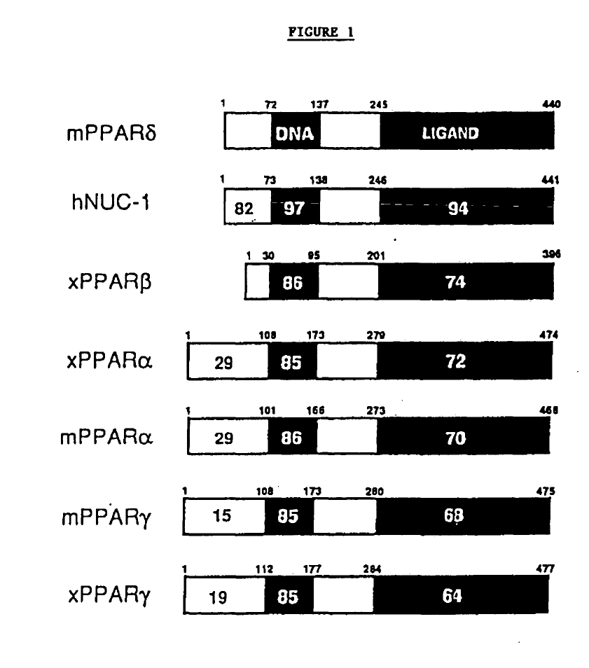

FIG. 1 presents a schematic comparison of the members of the PPAR gene family using mPPARδ as a reference. Comparisons among the different domains of the proteins are expressed as percent amino acid identity.

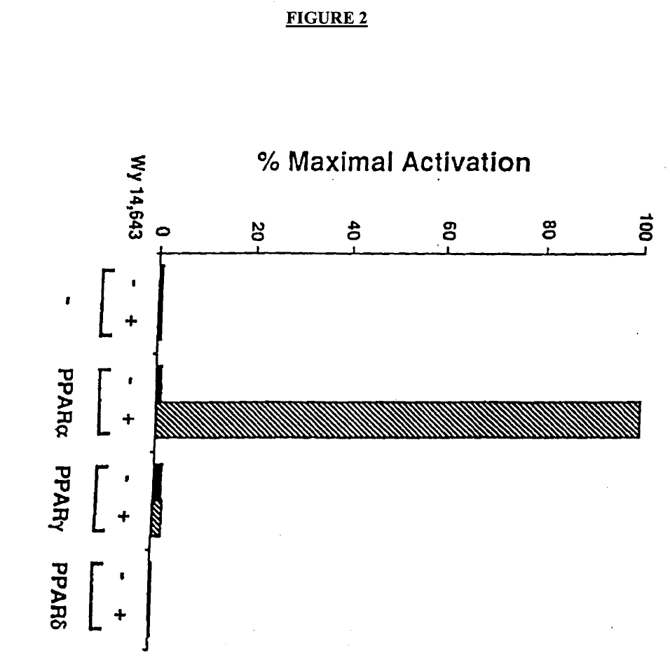

FIG. 2 demonstrates that PPARγ and PPARδ fail to respond to the peroxisome proliferator Wy 14,643. CV-1 cells were cotransfected with reporter plasmid PPRE₃-TK-LUC and either no receptor expression plasmid (−), CMX-PPARα, CMX-PPARγ, or CMX-PPARδ and then incubated in either the absence (−) or presence (+) of 5 μM Wy 14,643. Luciferase activities are expressed as percentages of the maximal response where 100% is the activity obtained with PPARα in the presence of 5 μM Wy 14,643.

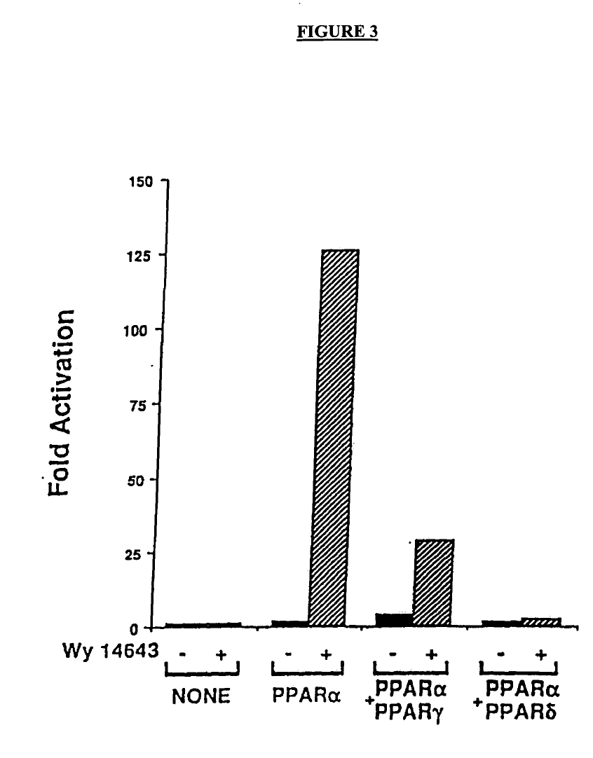

FIG. 3 illustrates the ability of PPARγ and PPARδ to repress PPARα-mediated responsiveness to Wy 14,643. CV-1 cells were cotransfected with reporter plasmid PPRE₃-TK-LUC and either no receptor expression plasmid (NONE) or CMX-PPARα (10 ng) in either the absence or presence of CMX-PPARγ (100 ng) or CMX-PPARδ (100 ng). Cells were then incubated in either the absence (−) or presence (+) of 5 μM Wy 14,643. Luciferase activities are presented as fold-activation relative to cells which were not transfected with receptor expression plasmid and were not treated with Wy 14,643.

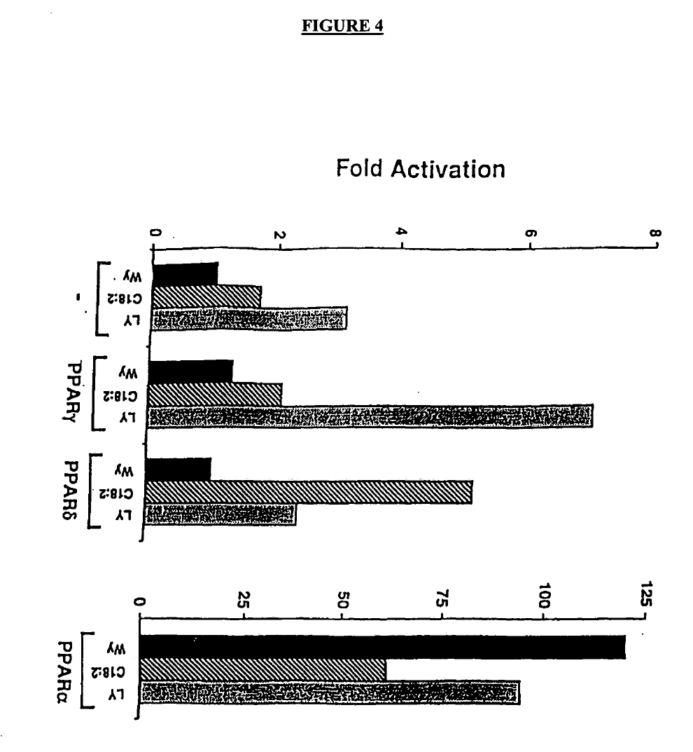

FIG. 4 demonstrates that PPAR isoforms are pharmacologically distinct. CV-1 cells were cotransfected with reporter plasmid PPRE₃-TK-LUC and either no receptor expression plasmid (−), CMX-PPARα, CMX-PPARγ, or CMX-PPARδ in either the absence or presence of 5 μM Wy 14,643 (WY), 3O μM linoleic acid (C18:2), or 30 μM LY-171883 (LY). Luciferase activities are presented as the fold activation achieved in compound-treated versus mock-treated cells. Similar results were obtained in triplicate in three independent experiments.

### DETAILED DESCRIPTION OF THE INVENTION

Two novel PPAR receptor subunits have been cloned and characterized. These novel γ and δ isoforms (subunits) together with the a subunit display marked differences in their responsiveness to peroxisome proliferators and fatty acids, as well as differences in their temporal and spatial patterns of expression. These observations suggest a broad role for the PPAR family during development and in adult physiology.

The existence of multiple PPAR isoforms with distinct expression patterns has been found to correlate with the fact that the three isoforms have different ligand specificities. Indeed, the PPAR isoforms are shown herein to be pharmacologically distinct. Thus, PPARα, PPARγ and PPARδ are most efficiently activated by Wy 14,643, LY-171883, and linoleic acid, respectively. Remarkably, Wy 14,643, which results in approximately 100-fold induction in reporter expression in the presence of PPARα, fails to activate either PPARγ or PPARδ.

With regard to this differential responsiveness to activators of peroxisome proliferation, the relationship among the PPAR isoforms may be analogous to that between the glucocorticoid and mineralocorticoid receptors (GR and MR, respectively). While both receptors can bind to the same response element, and both respond to mineralocorticoids and corticosteroids, MR and GR display differential sensitivities to aldosterone and specific glucocorticoids such as dexamethasone, respectively (Arriza et al., Neuron 1:887-900 (1988)). Thus, the ratio of these receptors to their ligands provides a means of determining tissue-specific expression of target genes. Similarly, the existence of multiple PPAR isoforms with overlapping ligand specificities may provide the means for tissue-specific regulation of gene expression by peroxisome proliferators and fatty acids.

In addition to their differential responsiveness to peroxisome proliferators, the three PPAR isoforms also display distinct yet overlapping expression patterns. As previously shown, PPARα mRNA is abundant in liver and kidney (Isseman and Green, supra Beck et al., Proc. R. Soc. Lond. 247:83-87 (1992)), tissues in which peroxisome proliferators result in dramatic increases in the numbers of peroxisomes and concomitant increases in peroxisomal β-oxidation (Nemali et al., supra). In contrast, the levels of PPARγ mRNA and PPARδ mRNA, which can act as dominant repressors of PPARα-mediated responsiveness to Wy 14,643, are bow in these tissues. Thus, a pattern emerges in which tissues that are most responsive to peroxisome proliferators such as Wy 14,643 are observed to express high amounts of PPARα mRNA and relatively low amounts of PPARγ mRNA and PPARδ mRNA. These data suggest that the ratio of the PPAR isoforms is likely to play a critical role in establishing the degree of responsiveness of tissues to specific peroxisome proliferators.

Widespread expression of PPARδ is observed in both the embryo and in adult tissues. This observation suggests that this isoform may play a general “housekeeping” role. In contrast, PPARγ is observed to be expressed almost exclusively in the adrenal and spleen. The expression of all three PPAR isoforms in the adrenal is particularly intriguing, since diseases which result in peroxisome dysfunction (e.g. adrenoleukodystrophy and Zellweger syndrome) cause gross morphological changes in adrenal cells and, eventually, adrenal deficiency. These observations suggest a critical role for peroxisomes in this tissue (Vamecq and Draye, supra). Interestingly, peroxisomes can be induced to proliferate in hamster adrenals in response to treatment with adrenocorticotropic hormone and corticosteroids (Black and Russo, Amer. J. Anatomy 159:85-120 (1980)), indicating the presence of adrenal-specific signaling pathway(s) for peroxisome proliferation. The differential expression of PPARγ in the adrenal suggests that this isoform may respond to an adrenal-enriched ligand.

Accordingly, in accordance with the present invention, there are provided isolated mammalian peroxisome proliferator-activated receptor subunit proteins of the α or δ subtype and functional fragments thereof.

As employed herein, the phrase “mammalian peroxisome proliferator-activated receptor subunit proteins of the γ or δ subtype” refers to isolated and substantially purified as well as recombinantly produced proteins which are members of the steroid/thyroid superfamily of receptors, and which mediate the pleiotropic effects of peroxisome proliferators (such as medium and bong-chain fatty acids). Such receptors participate in the formation of heterodimeric species with retinoid X receptors (RXRs) and comprise an amino-terminal domain, a DNA binding domain, and a ligand binding domain. Also contemplated within this definition are variants thereof encoded by mRNA generated by alternative splicing of a primary transcript.

Use of the terms “recombinantly produced”, “isolated” or “substantially pure” in the present specification and claims as a modifier of DNA, RNA, polypeptides or proteins means that the modified substances have been produced by the hand of man, and thus are separated from their native in vivo cellular environment. As a result of this human intervention, the recombinant/isolated/substantially pure DNAs, RNAs, polypeptides and proteins of the invention are useful in ways that the naturally occurring DNAs, RNAs, polypeptides or proteins are not, for example, in assays to identify selective drugs or compounds.

The novel receptors of the present invention also can be included as part of a panel of receptors which are screened to determine the selectivity of interaction of proposed agonists or antagonists of other steroid hormone receptors. Thus, a compound which is believed to interact selectively, for example, with the glucocorticoid receptor, should not have any substantial effect on any other receptors, including invention receptors. However, if such a proposed compound does interact with the invention receptors, then the probability of side effects caused by the activation of other receptors in addition to the target receptor, is clearly indicated. For example, the use of many drugs in the treatment of hormone-related disorders is currently restricted by side effects caused by the activation of “non-target” receptors. Employment of the invention receptors in a panel of receptors in a screen to determine the selectivity of interaction of potential bigands provides a means to identify receptor-specific ligands that are therapeutically superior than currently used ligands that cause unwanted side effects.

As used herein, the term splice variant refers to variant PPAR encoding nucleic acid(s) produced by differential processing of primary transcript(s) of genomic DNA, resulting in the production of more than one mRNA. cDNA derived from differentially processed primary transcript will encode PPAR receptor proteins that have regions of complete amino acid identity and regions having different amino acid sequences. Thus, the same genomic sequence can bead to the production of multiple, related mRNAs and corresponding proteins. Both the resulting mRNAs and proteins are referred to herein as “splice variants”.

Accordingly, also contemplated within the scope of the present invention are nucleic acids that encode mammalian PPAR receptor subunit proteins as defined above, but that by virtue a degenerate genetic code do not necessarily hybridize to the nucleic acids set forth in SEQ ID NOs: 1 or 3 under specific hybridization conditions. Nucleic acid fragments encoding invention receptor subunit proteins are capable of forming a functional heterodimer with one or more RXR receptor protein isoform(s). Typically, unless a PPAR receptor protein is encoded by mRNA that arises from alternative splicing (i.e., a splice variant), PPAR receptor encoding DNA and encoded protein share substantial sequence homology with at least one of the PPAR receptor-encoding DNAs and encoded proteins described herein. It is understood that DNA or RNA encoding a splice variant may share less than 90% overall sequence homology with the DNA or RNA provided herein, but include regions of nearly 100% homology to a DNA fragment described herein, and encode an open reading frame that includes start and stop codons and encodes a functional PPAR receptor protein.

Exemplary nucleic acid sequences encoding mammalian peroxisome proliferator-activated receptor subunit proteins of the γ subtype are represented by nucleotide sequences which encode substantially the same amino acid sequence as set forth in SEQ ID NO:2. Presently preferred sequences encode the same amino acid sequence as set forth in SEQ ID NO:2.

Exemplary nucleic acid sequences can alternatively be characterized as those nucleotide sequences which encode mammalian peroxisome proliferators-activated receptor subunit proteins of the γ subtype and hybridize under high stringency conditions to SEQ ID NO: 1.

Exemplary nucleic acid sequences encoding mammalian peroxisome proliferator-activated receptor subunit proteins of the δ subtype are represented by nucleotides which encode substantially the same amino acid sequence as set forth in SEQ ID NO:4. Presently preferred sequences encode the same amino acid sequence as set forth in SEQ ID NO:4.

Especially preferred sequences are those which have substantially the same nucleotide sequence as that set forth in SEQ ID NO:1.

Exemplary nucleic acid sequences can alternatively be characterized as those nucleotide sequences which encode mammalian peroxisome proliferators-activated receptor subunit proteins of the δ subtype and hybridize under high stringency conditions to SEQ ID NO:3.

Especially preferred nucleic acid sequences are those which have substantially the same nucleotide sequence as the coding sequences in SEQ ID NO:3.

The phrase “stringency of hybridization” is used herein to refer to conditions under which polynucleic acid hybrids are stable. As known to those of skill in the art, the stability is reflected in the melting temperature (Tₘ) of the hybrids. Tₘ can be approximated by the formula:
81.5° C.−16.6(log₁₀[Na⁺])+0.41(%G+C)−600/1,
where 1 is the length of the hybrid in number of nucleotides. Tₘ decreases approximately 1-1.5° C. with every 1% decrease in sequence homology. In general, the stability of a hybrid is a function of sodium ion concentration and temperature. Typically, the hybridization reaction is initially performed under conditions of low stringency, followed by washes of varying, but higher, stringency. Reference to hybridization stringency relates to such washing conditions. Thus, as used herein:
(1) HIGH STRINGENCY refers to conditions that permit hybridization of only those nucleic acid sequences that form stable hybrids in 0.01 8M NaCl at 65° C. (i.e., if a hybrid is not stable in 0.01 8M NaCl at 65° C., it will not be stable under high stringency conditions, as contemplated herein). High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C.;
(2) MODERATE STRINGENCY refers to conditions that permit hybridization in 50% formamide, sx Denhart's solution, 5×SSPE, 0.2% SDS at 42° C., followed by washing in 0.2×SSPE, 0.2% SDS, at 65° C.; and
(3) LOW STRINGENCY refers to conditions that permit hybridization in 10% formamide, 5× Denhart's solution, 6×SSPE, 0.2% SDS at 42° C., followed by washing in 1×SSPE, 0.2% SDS, at 50° C.
   It is understood that these conditions may be varied using a variety of buffers and temperatures well known to skilled artisans.

As used herein, the phrase “substantial sequence homology” refers to nucleotide sequences which share at least about 90% identity, and amino acid sequences which typically share more than 95% amino acid identity. It is recognized, however, that proteins (and DNA or mRNA encoding such proteins) containing less than the above-described level of homology arising as splice variants or that are modified by conservative amino acid substitutions (or substitution of degenerate codons) are contemplated to be within the scope of the present invention.

As used herein, the phrase “substantially the same” refers to nucleotide sequences, ribonucleotide sequences, or amino acid sequences, that have slight and non-consequential sequence variations from the actual sequences disclosed herein. Species that are “substantially the same” are considered to be equivalent to the disclosed sequences, and as such are within the scope of the appended claims. In this regard, “slight and non-consequential sequence variations” mean that sequences that are substantially the same as invention sequences disclosed and claimed herein, are functionally equivalent to the sequences disclosed and claimed herein. Functionally equivalent sequences will function in substantially the same manner to produce substantially the same results as the nucleic acid and amino acid sequences disclosed and claimed herein. Specifically, functionally equivalent nucleic acids encode proteins that have conservative amino acid variations, such as substitution of a non-polar residue for another non-polar residue or a charged residue for a similarly charged residue. These changes are recognized by those of skill in the art as modifications that do not substantially alter the tertiary structure of the protein.

Fragments of invention nucleic acid sequences are useful as hybridization probes, wherein such fragments comprise at least 14 contiguous nucleotides of the above-described nucleic acids, and wherein the fragment is labeled with a detectable substituent. Suitable detectable substituents can be readily determined by those of skill in the art, and include such species as radiolabeled molecules, fluorescent molecules, enzymes, ligands, and the like.

As used herein, a probe is single- or doublestranded DNA or RNA that has a sequence of nucleotides that includes at least 14 contiguous bases that are the same as (or the complement of) any 14 or more contiguous bases set forth in SEQ ID NOs:1 or 3. Preferred regions for the construction of probes include those regions predicted to encode a DNA binding domain. Such regions are preferred because they are most highly conserved among members of the steroid/thyroid superfamily of receptors.

As a particular application of the invention sequences, genetic screening can be carried out using the nucleic acid sequences of the invention as probes. Thus, nucleic acid samples from patients having conditions suspected of involving alteration/modification of any one or more of the PPAR receptor subtypes can be screened with appropriate probes to determine if abnormalities exist with respect to the endogenous PPAR receptor proteins.

In accordance with yet another embodiment of the present invention, there are provided vectors comprising nucleic acid sequences, as well as cells and vectors containing such sequences. Such host cells, including bacterial, yeast and mammalian cells can be used for expressing invention nucleic acids to produce PPAR receptor protein(s). Incorporation of cloned DNA into a suitable expression vector, transfection of eukaryotic cells with a plasmid vector or a combination of plasmid vectors, each encoding one or more distinct genes, and selection of transfected cells are well known in the art (see, e.g., Sambrook et al. (1989) Molecular Cloning A Laboratory Manual, Second Edition, Cold Spring Harbor Laboratory Press). Heterologous DNA may be introduced into host cells by any method known to those of skill in the art, such as transfection by CaPO₄ precipitation with a vector encoding the heterologous DNA (see, e.g., Wigler et al. (1979) Proc. Natl. Acad. Sci. 76:1373-1376), DEAE-dextran, electroporation, microinjection, or lipofectamine (GIBCO BRL #18324-012). Transfected host cells can then be cultured under conditions whereby the receptor subunit protein(s) encoded by the DNA is (are) recombinantly expressed.

The present invention further provides a mammalian peroxisome proliferator-activated receptor, expressed recombinantly in a host cell. The receptor comprises at least one PPAR subunit, wherein the PPAR subunit is PPARγ or PPARδ, and at least one retinoid X receptor isoform. The invention receptor has the ability to repress PPARα-mediated responses activated by Wy 14,643.

Also provided by the present invention are mammalian peroxisome proliferator-activated subunit proteins, expressed recombinantly in a host cell wherein the receptor subunits have substantially the same amino acid sequence as set forth in SEQ ID NOs: 2 or 4.

In accordance with still another embodiment of the present invention, there is provided a method for the recombinant production of mammalian peroxisome proliferator-activated receptor proteins comprising at least one PPAR subunit of the γ or δ subtype, or functional fragments thereof. Such method comprises expressing the above-described nucleic acid(s) in a suitable host cell.

In accordance with still another embodiment of the present invention, there is provided a method to identify clones encoding mammalian peroxisome proliferatoractivated receptor subunit proteins of the γ or δ subtype, or functional fragments thereof. Such method comprises screening a genomic or cDNA library with an invention nucleic acid probe under low stringency hybridization conditions, and identifying those clones which display a substantial degree of hybridization to said fragment.

Nucleic acids encoding mammalian peroxisome proliferator-activated receptor subunit protein of the γ or δ subtype, or functional fragments thereof may be isolated by screening suitable human cDNA or human genomic libraries under suitable hybridization conditions with nucleic acids disclosed herein (including nucleotide sequences derived from SEQ ID NOs:1 or 3). Suitable libraries can be prepared from appropriate tissue samples, e.g., brain tissue, heart tissue, intestinal tissue, kidney tissue, liver tissue, spleen tissue, and the like. The library can be screened with nucleic acid including substantially the entire receptor-encoding sequence thereof, or the library may be screened with a suitable probe, as described above.

After screening the library, positive clones are identified by means of a hybridization signal; the identified clones are characterized by restriction enzyme mapping and/or DNA sequence analysis, and then examined, by comparison with the sequences set forth herein to ascertain whether they encode a complete PPAR receptor subunit protein (i.e., if they include translation initiation and termination codons). If the selected clones are incomplete, they may be used to rescreen the same or a different library to obtain overlapping clones. If the library is genomic, then the overlapping clones may include exons and introns. If the library is a cDNA library, then the overlapping clones will include an open reading frame. In both instances, complete clones may be identified by comparison with the DNA and encoded proteins provided herein.

The ligand-binding domain (LBD) of nuclear hormone receptors is a complex multifunctional unit containing subdomains for dimerization, transcriptional suppression and hormone-induced transactivation (Forman and Samuels, Mol. Endocrinol. 4:1293-1301(1990)). The dimerization domain includes a series of heptad repeats flanked by sequences required for ligand binding. Thus, the dimerization domain is embedded within the larger LBD. This structural arrangement raises the possibility that dimerization may serve as an allosteric modulator of ligand binding and transactivation. It has previously been shown that the Drosophila ecdysone receptor (EcR) acquires ligand binding activity after heterodimerization with USP (Drosophila homolog of RXR; see Yao et al., in Nature 366:476-479 (1993)). Thus, differential interactions among receptor LBDs can either restrict, redirect or lead to an acquisition of new ligand binding phenotypes.

It has recently been shown that PPARα binds to its cognate response elements as a heterodimer with the RXR (see Kliewer et al., supra, Gearing et al., supra, or Keller et al., supra). The resulting PPARα-RXR complex can respond to both peroxisome proliferators and 9-cis retinoic acid (see Kliewer et al., (1992), supra). It has now been found that PPARγ and PPARδ also cooperate with RXR in the formation of heterodimers, and in binding to DNA as heterodimers. Ultimately, the regulation of peroxisome physiology is likely a consequence of a complex interplay among the multiple PPAR and RXR isoforms and the ligands for these receptors.

In accordance with the present invention, there are provided combinations of receptors comprising at least two different members of the steroid/thyroid superfamily of receptors, wherein one receptor is either PPARγ or PPARδ, and wherein said receptors are associated in the form of a multimer, preferably a heterodimer. A particularly preferred combination of receptors is a heterodimer comprising either PPARγ or PPARδ and a subtype of RXR.

Combinations contemplated by the present invention can broadly be referred to as “multimeric species,” which is intended to embrace all of the various oligomeric forms in which members of the steroid/thyroid superfamily of receptors (including fragments thereof comprising the dimerization domains thereof) are capable of associating in combination with either PPARγ or PPARδ. Thus, reference to “combinations” of steroid receptors or “multimeric” forms of steroid receptors includes homodimeric combinations of a single PPARγ or PPARδ receptor (including fragments thereof comprising the dimerization domains thereof), heterodimeric combinations of either a PPARγ or PPARδ receptor and another different receptor (including fragments thereof comprising the dimerization domains thereof), homotrimeric combinations of a single PPARγ or PPARδ receptor (including fragments thereof comprising the dimerization domains thereof), heterotrimeric combinations of two or three different receptors including PPARγ or PPARδ (including fragments thereof comprising the dimerization domains thereof) homotetrameric combinations of a single PPARγ or PPARδ receptor (including fragments thereof comprising the dimerization domains thereof), heterotetrameric combinations of two or more different receptors including PPARγ or PPARδ (including fragments thereof comprising the dimerization domains thereof), and the like.

As employed herein, the phrase “members of the steroid/thyroid superfamily of receptors” (also known as “nuclear receptors” or “intracellular receptors”) refers to hormone binding proteins that operate as ligand-dependent transcription factors, including identified members of the steroid/thyroid superfamily of receptors for which specific ligands have not yet been identified (referred to hereinafter as “orphan receptors”). These hormone binding proteins have the intrinsic ability to bind to specific DNA sequences. Following binding, the transcriptional activity of target gene (i.e., a gene associated with the specific DNA sequence) is modulated as a function of the ligand bound to the receptor.

The DNA-binding domains of all of these nuclear receptors are related, consisting of 66-68 amino acid residues, and possessing about 20 invariant amino acid residues, including nine cysteines. A member of the superfamily can be identified as a protein which contains the above-mentioned invariant amino acid residues, which are part of the DNA-binding domain of such known steroid receptors as the human glucocorticoid receptor (amino acids 421-486), the estrogen receptor (amino acids 185-250), the mineralocorticoid receptor (amino acids 603-668), the human retinoic acid receptor (amino acids 88-153). The highly conserved amino acids of the DNA-binding domain of members of the superfamily are well-known as set forth, for example in PCT WO 94/01558. Thus, the DNA-binding domain is a minimum of 66 amino acids in length, but can contain several additional residues.

Exemplary members of the steroid/thyroid superfamily of receptors contemplated for use in the practice of the present invention (including the various isoforms thereof) include steroid receptors such as mineralocorticoid receptor, progesterone receptor, androgen receptor, vitamin D₃ receptor, and the like; plus retinoid receptors, such as the various isoforms of RAR (e.g., RARα, RARβ, or RARγ), the various isoforms of RXR (e.g., RXRα, RXRβ, or RXRγ), and the like; thyroid receptors, such as TRα, TRβ, and the like; as well as other gene products which, by their structure and properties, are considered to be members of the superfamily, as defined hereinabove, including the various isoforms thereof. Examples of orphan receptors include HNF4 [see, for example, Sladek et al., in Genes & Development 4: 2353-2365 (1990)], the COUP family of receptors [see, for example, Miyajima et al., in Nucleic Acids Research 16: 11057-11074 (1988), and Wang et al., in Nature 340: 163-166 (1989)], COUP-like receptors and COUP homologs, such as those described by Mlodzik et al., in Cell 60: 211-224 (1990) and Ladias et al., in Science 251:561 -565 (1991), the ultraspiracle receptor example, [see, for example, Oro et al., in Nature 347: 298-301 (1990)], and the like. Presently preferred members of the superfamily for use in the practice of the present invention are the various isoforms of RXR (e.g., RXRα, RXRβ, or RXRγ).

The formation of multimeric (e.g., heterodimeric) species can modulate the ability of the first receptor to trans-activate transcription of genes maintained under expression control in the presence of ligand for said first receptor. The actual effect on activation of transcription (i.e., enhancement or repression of transcription activity) will vary depending on the receptor species which is combined with either a PPARγ or PPARδ receptor to form the multimeric species, as well as on the response element with which the multimeric species interacts.

In accordance with the present invention, there are provided multimeric receptor species which belong to the steroid/thyroid superfamily of receptors, comprising at least the dimerization domain of at two different members of the steroid/thyroid superfamily of receptors, wherein one of the members is selected from the invention PPARγ or PPARδ.

As employed herein, the tern “dimerization domain” of a member of the steroid/thyroid superfamily of receptors refers to that portion of the receptor which is believed to be involved in the formation of multimeric receptor species. This domain typically comprises the carboxy-terminal portion of the receptor, i.e., that portion of a receptor which is 3′ with respect to the DNA-binding domain of the receptor. See, e.g., Evans, in Science 240:889-895 (1988), and Forman and Samuels, Mol. Endocrinol. 4:1293-1301 (1990). Presently preferred members of the superfamily for use in deriving the dimerization domain are the various isoforms of RXR (e.g., RXRα, RXRβ, or RXRγ).

In accordance with the present invention, there are also provided heterodimer complexes comprising either PPARγ or PPARδ and a silent partner therefor.

As employed herein, the term “silent partner” refers to members of the steroid/thyroid superfamily of receptors which are capable of forming heterodimeric species with either PPARγ or PPARδ, wherein the silent partner of the heterodimer does not have any ligand bound to the ligand-binding domain (LBD) when the silent partner is complexed with a PPAR subtype (i.e., only the PPAR copartner of the heterodimer binds ligand). Presently preferred silent partners for use in the practice of the present invention are the various isoforms of RXR (e.g., RXRα, RXRβ, or RXRγ).

In accordance with a further embodiment of the present invention, there is provided a method for screening compounds to determine those which bind to mammalian peroxisome proliferator-activated receptor proteins comprising at least one PPAR subunit of the γ or δ subtype, or functional fragments thereof. Such method comprises employing receptor protein(s) of the invention in a binding assay, which comprises, contacting receptor protein(s) of the invention with test compound, and identifying those compounds which bind to invention receptor protein(s).

In accordance with a still further embodiment of the present invention, there is provided a bioassay for evaluating whether test compounds are agonists for receptor proteins of the invention, or functional modified forms of said receptor protein(s). Such bioassay comprises:
(1) contacting suitable host cells expressing said receptor protein with test compound under physiological conditions, wherein said host cells contain DNA encoding a reporter protein, wherein said DNA is operatively linked to a PPAR-response element;
(2) monitoring said host cells for expression of reporter gene, wherein expression of reporter protein reflects transcriptional activity of the receptor protein and, therefore, the presence of an activated receptor-ligand complex.

In accordance with yet another embodiment of the present invention, there is provided a bioassay for evaluating whether test compounds are antagonists for receptor proteins of the invention, or functional modified forms of said receptor protein(s). Such bioassay comprises:

contacting suitable host cells with
(i) increasing concentrations of at least one compound whose ability to inhibit the transcription activation activity of agonists of mammalian peroxisome proliferator-activated receptor proteins of the γ or δ subtype is sought to be determined, and
(ii) a fixed concentration of at least one agonist for said receptor protein(s) or functional modified forms thereof,
wherein suitable test cells express mammalian peroxisome proliferator-activated receptor proteins comprising at least one PPAR subunit of the γ or δ subtype and DNA encoding a reporter protein, wherein said DNA is operatively linked to a PPAR-response element; and thereafter

assaying for evidence of transcription of said reporter gene in said cells as a function of the concentration of said compound in said culture medium, thereby indicating the ability of said compound to inhibit activation of transcription by agonists of mammalian peroxisome proliferator-activated receptor proteins comprising at least one PPAR subunit of the γ or δ subtype.

In accordance with a still further embodiment of the present invention, there is provided a method for identifying ligands selective for heterodimers comprising either PPARγ or PPARδ and a silent partner therefor. Such method comprises

comparing the level of expression of reporter when cells containing a reporter construct, either PPARγ or PPARδ and silent partner therefor are exposed to test compound, relative to the level of expression of reporter when cells containing a reporter construct, either PPARγ or PPARδ and a member of the steroid/thyroid superfamily which is not a silent partner therefor are exposed to test compound, and

selecting those compounds which activate only the combination of either PPARγ or PPARδ and silent partner therefor.

In accordance with yet another embodiment of the present invention, there are provided antibodies generated against the invention proteins. Such antibodies can be employed for studying receptor tissue localization, subunit composition, structure of functional domains, as well as in diagnostic applications, therapeutic applications, and the like. Preferably, for therapeutic applications, the antibodies employed will be monoclonal antibodies.

The above-described antibodies can be prepared employing standard techniques, as are well known to those of skill in the art, using the invention receptor proteins or portions thereof as antigens for antibody production. Both anti-peptide and anti-fusion protein antibodies can be used Trends Pharmacol Sci. vol. 12:338-343; Current Protocols in Molecular Biology (Ausubel et al., eds.) John Wiley and Sons, New York (1989)]. Factors to consider in selecting portions of the invention receptor protein subunit sequences for use as immunogen (as, for example, a synthetic peptide or a recombinantly produced bacterial fusion protein) include antigenicity, accessibility (i.e., internal or external domains), uniqueness to the particular protein subunit, and the like.

The availability of sequence-specific antibodies enables use of immunohistochemical techniques to monitor the distribution and expression density of various protein subunits (e.g., in normal versus diseased brain tissue). Such antibodies can also be employed for diagnostic and therapeutic applications.

In accordance with yet another embodiment of the present invention, there are provided methods for modulating processes mediated by mammalian peroxisome proliferator-activated receptor proteins comprising at least one PPAR subunit of the γ or δ subtype. Such methods comprise contacting mammalian peroxisome proliferatoractivated receptor proteins of the γ or δ subtype with an effective, modulating amount of agonist, antagonist or antibody according to the present invention.

The antibodies, agonists and/or antagonists of the invention can be administered to a subject employing standard methods, such as, for example, by intraperitoneal, intramuscular, intravenous, or subcutaneous injection, implant or transdermal modes of administration, and the like. One of skill in the art can readily determine dose forms, treatment regiments, etc, depending on the mode of administration employed.

Processes which are mediated by mammalian peroxisome proliferator-activated receptor proteins of the γ or δ subtype include, for example, macrophage production in the spleen which is believed to be important in atherosclerosis.

The invention will now be described in greater detail with reference to the following non-limiting examples.

### EXAMPLE 1

### Screening of cDNA Libraries

PPARγ was isolated by screening an adult mouse liver λZAP cDNA library (Stratagene) with a synthetic oligonucleotide (GGNTTYCAYTAYGGNGTNCAYCG; SEQ ID NO: 5) under conditions previously described by Blumberg et al., in Proc. Natl. Acad. Sci. USA 89:2321-2325 (1992). This oligonucleotide is a mixture of all possible DNA sequences encoding the amino acid sequence GFHYGVHA (SEQ ID NO:6), a sequence present in the loop of the first zinc finger in the Xenopus PPARα PPARβ and PPARγ isoforms.

PPARδ was isolated by screening an E6.5 mouse λZAPII cDNA library (a gift of D. E. Weng and J. D. Gerhart, Johns Hopkins University) under low stringency conditions with a cDNA fragment encoding the human retinoic acid receptor aDNA binding domain (Mangelsdorf et al., Nature 345:224-229 (1990)). In both screens, positive clones were converted to plasmids by the automatic excision process.

### EXAMPLE 2

### Cotransfection Assay

The mammalian expression vectors pCMX-PPARα, pCMX-PPARγ and pCMX-PPARδ were constructed by inserting the cDNA inserts of PPARα, PPARγ, and PPARδ into pCMX as previously described by Umesono et al., in Cell 65:1255-1266 (1991)). Construction of the reporter PPRE₃-TK-LUC has also been previously described by Kliewer et al., (1992) supra. Cotransfection assays in CV-1 cells were done in 48 well plates using N-[1-(2,3-dioleoyloxy)-propyl [N,N,N-trimethyl ammonium methyl sulfate (DOTAP) according to the manufacturer's instructions (Boehringer Mannheim). Transfections contained 10 ng of receptor expression plasmid vector, 20 ng of the reporter PPRE₃-TK-LUC, 60 ng of pCMX-βGAL (β-galactosidase) as an internal control, and 210 ng of carrier plasmid pGEM. Cells were incubated in the presence of DOTAP for 8 hours, washed, and incubated in the presence of peroxisome proliferators or fatty acids for 36 hours. Cell extracts were prepared and assayed for luciferase and β-galactosidase activity as previously described (Umesono, supra). All experimental points were done in triplicate.

### EXAMPLE 3

### Northern Analysis

Preparation of poly(A)⁺RNA from rat tissues and Northern analysis were performed as previously described (Mangelsdorf et al., supra). Thus, Northern blot analysis of PPAR mRNA was carried out employing adult and embryonic tissue. Adult male rat tissues and mouse embryos from gestation day 10.5 to 18.5 were employed. The exposure time for each of the blots was 48 hours. The sizes of the transcripts, based on RNA size markers, were 8.5 kb (PPARα), 1.9 kb (PPARγ), and 3.5 kb (PPARδ).

### EXAMPLE 4

### DNA Binding Assays

Gel mobility shift assays were performed as previously described by Kliewer et al. (1992) supra. PPARα, PPARγ, PPARδ, RXRβ, RXR,δ and RXRγ were synthesized in vitro using the TNT coupled transcription/translation system (Promega) according to the manufacturer's instructions.

### EXAMPLE 5

### Isolation of Three Murine PPAR Isoforms

The function of peroxisome proliferators has been most extensively studied in rodents, where treatment with these compounds results in marked increases in peroxisome size and number and concomitant increases in the expression of the genes encoding the enzymes of the peroxisomal β-oxidation pathway. To gain insight into the function of PPAR isoforms, mouse embryonic and adult liver libraries were screened for PPARα-related gene products. In addition to PPARα, two types of PPARα-related clones were isolated.

The first clone encodes a 475-amino acid protein that is 56% identical to mouse (m)PPARα and 76% identical to Xenopus (x)PPARγ. Since this clone is 97% and 84% identical to the DNA binding and ligand binding domains of xPPARγ, respectively, it is designated mPPARγ (see SEQ ID NOs:1 and 2).

The second clone encodes a 440-amino acid protein that is closely related to NUC-1 (see SEQ ID NOs:3 and 4, and FIG. 1), a PPARα-related receptor recently isolated from a human osteosarcoma library (see Schmidt et al., in Mol. Endo. 6:1634-1641 (1992)). Since this second clone is not highly homologous to any of the previously identified PPAR isoforms (i.e., mPPARα, xPPARα, xPPARβ or xPPARγ, see FIG. 1), it appears to represent a novel receptor, and is, therefore, designated mPPARδ. Of the approximately 50 positives characterized during the course of screening, no mouse homolog of xPPARβ was identified.

### EXAMPLE 6

### PPARα, PPARγ, and PPARδ are Differentially Expressed in the Adult and Embryo

The expression patterns of the murine PPAR isoforms were examined in the embryo and adult. Northern analysis of poly(A)⁺ RNA isolated from adult male rat tissues revealed differential yet overlapping patterns of expression of the three isoforms. Both PPARα and PPARδ are widely expressed, with PPARα message levels highest in the liver, kidney, heart, and adrenal, and PPARδ message highest in the heart, adrenal, and intestine. In contrast, PPARγ displays a more restricted distribution pattern, with abundant expression in only the adrenal and spleen, although message is also detectable in the heart, kidney, and intestine.

The developmental expression of the PPAR isoforms was also examined through Northern analysis of whole mouse embryo RNA. PPARα and PPARγ displayed similar expression patterns during mouse embryogenesis, with message first appearing at day 13.5 postconception and increasing until birth. In contrast, PPARδ message was abundant at all the embryonic time points tested, suggesting a broad role for this isoform during development. Thus, the PPAR isoforms are seen to be differentially expressed in both the embryo and the adult.

### EXAMPLE 7

### Evidence for Pharmacological Differences Between PPARα, PPARγ and PPARβ

The relatively high degree of conservation within the ligand binding domains of PPARα, PPARγ and PPARδ suggested that these PPAR isoforms might respond to the same activators. Accordingly, each of the PPAR isoforms was first tested for responsiveness to Wy 14,643, a peroxisome proliferator and potent activator of PPARα (Reddy and Laiwani, Crit. Rev. Toxicol. 12:1-58 (1983)). Cotransfection of PPARα expression plasmid resulted in a dramatic (>100-fold) increase in activation of a reporter construct containing three copies of the acyl-CoA oxidase PPRE (AOX-PPRE) upstream of the thymidine kinase promoter driving luciferase expression (PPRE₃-TK-LUC) in response to Wy 14,643 (FIG. 2).

In contrast, no activation of reporter expression was seen in the presence of Wy 14,643 upon cotransfection of PPARγ or PPARδ expression plasmids (FIG. 2). This lack of activation is unlikely to reflect differences in binding site specificity, as each of the PPAR isoforms bound efficiently to the AOX-PPRE as a heterodimer with RXR (as determined by gel mobility shift assays done using in vitro synthesized PPARα, PPARγ, and PPARδ, and/or RXRγ, and ³²P-labeled AOX-PPRE oligonucleotide). Additional experiments revealed that overexpression of PPARγ and PPARδ interfered with the ability of PPARα to activate through the AOX-PPRE (FIG. 3). Thus, both PPARγ and PPARδ are expressed and can function as dominant repressors of PPARα-mediated responsiveness to Wy 14,643.

Since no activation of PPARγ and PPARδ was detected with Wy 14,643, other potential activators were tested, including a broad spectrum of peroxisome proliferators and fatty acids. As shown in FIG. 4, significant activation of PPARγ was obtained upon treatment with LY-171883, a leukotriene antagonist and peroxisome proliferator which lacks the carboxyl group typically found in this class of compounds (Foxworthy and Eacho, Biochem. Pharmacology 42:1487-1491 (1991)). Conversely, no activation of PPARγ was seen in the presence of linoleic acid (FIG. 4).

In contrast to the results obtained with PPARγ, PPARδ was activated in the presence of linoleic acid, but was not activated upon treatment with LY-171883. Both LY-171883 and linoleic acid are strong activators of PPARα (FIG. 4). Interestingly, each of the three PPAR isoforms was activated with a distinct rank order of efficacy by these compounds:

PPARα.
Wy 14,643>L,Y-171883>linoleic acid;

PPARγ.
LY 171883>linoleic acid>Wy 14,643;

PPARδ:
linoleic acid>LY-171883 and Wy 14,643. See FIG. 4. These data provide evidence that PPARγ and PPARδ can function as regulated activators of gene expression and that the three PPAR isoforms are pharmacologically distinct.

While the invention has been described in detail with reference to certain preferred embodiments thereof, it will be understood that modifications and variations are within the spirit and scope of that which is described and claimed.

That which is claimed is:

1. An isolated mammalian peroxisome proliferator-activated receptor subunit protein of the gamma subtype (PPAR-γ), or functional fragments thereof.

2. A protein according to claim 1 wherein said protein is characterized by:
the ability to repress PPARα-mediated responses activated by Wy 14,643;
being activated by LY-171883, but not linoleic acid; and
being encoded by nucleic acid which hybridizes under high stringency conditions to a nucleic acid which encodes the amino acid sequence set forth in SEQ ID NO:2.

3. A protein according to claim I wherein said PPAR-γ has more than 95% amino acid identity with SEQ ID NO: 2.

4. A protein according to claim 1 having an amino acid sequence substantially the same as set forth in SEQ ID NO:2.

5. A protein according to claim 1 having the same amino acid sequence as set forth in SEQ ID NO:2.

6. An isolated mammalian peroxisome proliferator-activated receptor subunit protein of the delta subtype (PPAR-δ), or functional fragments thereof.

7. A protein according to claim 6 wherein said protein is characterized by:
the ability to repress PPARα-mediated responses activated by Wy 14,643;
being activated by LY-171883, but not linoleic acid; and
being encoded by nucleic acid which hybridizes under high stringency conditions to a nucleic acid which encodes the amino acid sequence set forth in SEQ ID NO:4.

8. A protein according to claim 6 wherein said PPAR-δ has more than 95% amino acid identity with SEQ ID NO: 4.

9. A protein according to claim 6 having an amino acid sequence substantially the same as set forth in SEQ ID NO:4.

10. A protein according to claim 6 having the same amino acid sequence as set forth in SEQ ID NO:4.

11. A heterodimer complex comprising a receptor according to claim 1 and an isoform of RXR.

12. A heterodimer complex according to claim 11 wherein said isoform of RXR is selected from the group consisting of RXRα, RXRβ and RXRγ.

13. A heterodimer complex comprising a receptor according to claim 6 and an isoform of RXR.

14. A heterodimer complex according to claim 13 wherein said isoform of RXR is selected from the group consisting of RXRα, RXRβ and RXRγ.

15. An antibody generated against the receptor of claim 1.

16. An antibody generated against the receptor of claim 6.

17. A method for identifying compounds potentially useful for the treatment of diseases modulated by a PPAR-γ according to claim 1, said method comprising determining whether a compound interacts directly with said PPAR-γ, thereby identifying compounds that interact directly with PPAR-γ as useful for the treatment of diseases modulated by PPAR-γ.

18. A method for identifying compounds potentially useful for the treatment of diseases modulated by a PPAR-γ according to claim 1, said method comprising determining whether a compound activates said PPAR-γ, thereby identifying compounds that activate PPAR-γ as useful for the treatment of diseases modulated by PPAR-γ.

19. A method for identifying compounds potentially useful for the treatment of diseases modulated by a PPAR-δ according to claim 6, said method comprising determining whether a compound interacts directly with said PPAR-δ, thereby identifying compounds that interact directly with PPAR-δ as useful for the treatment of diseases modulated by PPAR-δ.

20. A method for identifying compounds potentially useful for the treatment of diseases modulated by a PPAR-δ according to claim 6, said method comprising determining whether a compound activates said PPAR-δ, thereby identifying compounds that activate PPAR-δ as useful for the treatment of diseases modulated by PPAR-δ.


SEQUENCE LISTING (6 sequences)

SEQ ID NO: 1 - DNA (cDNA), 2005 bp; both-stranded, linear
  CDS 352..1776
atcgaatcccgcgccccaggcgctgccgctctgagtgcgacgggccccgcctggccggccggaggacgcggaagaagagacctggggcgctgcctggggtattgggtcgcgcgcagtgaggggaccgagtgtgacgacaaggtgaccgggctgaggggacgggctgaggagaagtcacactctgacaggagcctgtgagaccaacagcctgacggggtctcggttgaggggacgcgggctgagaagtcacgttctgacaggactgtgtgacagacaagatttgaaagaagcggtgaaccactgatattcaggacatttttaaaaacaagactaccctttactgaaattaccatggttgacacagagatgccattctggcccaccaacttcggaatcagctctgtggacctctccgtgatggaagaccactcgcattcctttgacatcaagccctttaccacagttgatttctccagcatttctgctccacactatgaagacattccattcacaagagctgacccaatggttgctgattacaaatatgacctgaagctccaagaataccaaagtgcgatcaaagtagaacctgcatctccaccttattattctgaaaagacccagctctacaacaggcctcatgaagaaccttctaactccctcatggccattgagtgccgagtctgtggggataaagcatcaggcttccactatggagttcatgcttgtgaaggatgcaagggttttttccgaagaaccatccgattgaagcttatttatgataggtgtgatcttaactgccggatccacaaaaaaagtagaaataaatgtcagtactgtcggtttcagaagtgccttgctgtggggatgtctcacaatgccatcaggtttgggcggatgccacaggccgagaaggagaagctgttggcggagatctccagtgatatcgaccagctgaacccagagtctgctgatctgcgagccctggcaaagcatttgtatgactcatacataaagtccttcccgctgaccaaagccaaggcgagggcgatcttgacaggaaagacaacggacaaatcaccatttgtcatctacgacatgaattccttaatgatgggagaagataaaatcaagttcaaacatatcacccccctgcaggagcagagcaaagaggtggccatccgaatttttcaagggtgccagtttcgatccgtagaagccgtgcaagagatcacagagtatgccaaaaatatccctggtttcattaaccttgatttgaatgaccaagtgactctgctcaagtatggtgtccatgagatcatctacacgatgctggcctccctgatgaataaagatggagtcctcatctcagagggccaaggattcatgaccagggagttcctcaaaagcctgcggaagccctttggtgactttatggagcctaagtttgagtttgctgtgaagttcaatgcactggaattagatgacagtgacttggctatatttatagctgtcattattctcagtggagaccgcccaggcttgctgaacgtgaagcccatcgaggacatccaagacaacctgctgcaggccctggaactgcagctcaagctgaatcacccagagtcctctcagctgttcgccaaggtgctccagaagatgacagacctcaggcagatcgtcacagagcacgtgcagctactgcatgtgatcaagaagacagagacagacatgagccttcaccccctgctccaggagatctacaaggacttgtattagcaggaaagtcccacccgctgacaacgtgttccttctattgattgcactattattttgagggaaaaaaatctgacacctaagaaatttactgtgaaaaagcatttaaaaacaaaaagttttagaacatgatctattttatgcatattgtttataaagatacatttacaatttacttttaatattaaaaattaccacattataaaaaaaaaaaaaaaaaaggaattcc

SEQ ID NO: 2 - protein, 475 aa
MVDTEMPFWPTNFGISSVDLSVMEDHSHSFDIKPFTTVDFSSISAPHYEDIPFTRADPMVADYKYDLKLQEYQSAIKVEPASPPYYSEKTQLYNRPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKNIPGFINLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKVLQKMTDLRQIVTEHVQLLHVIKKTETDMSLHPLLQEIYKDLY

SEQ ID NO: 3 - DNA (cDNA), 2012 bp; both-stranded, linear
  CDS 263..1582
gaattccctggggattaatgggaaaagttttggcaggagctgggggattctgcggagcctgcgggacggcggcagcggcgcgagaggcggccgggacagtgctgtgcagcggtgtgggtatgcgcatgggactcactcagaggctcctgctcactgacagatgaagacaaacccacggtaaaggcagtccatctgcgctcagacccagatggtggcagagctatgaccaggcctgcagcgccacgccaagtgggggtcagtcatggaacagccacaggaggagacccctgaggcccgggaagaggagaaagaggaagtggccatgggtgacggagccccggagctcaatgggggaccagaacacacgcttccttccagcagctgtgcagacctctcccagaattcctccccttcctccctgctggaccagctgcagatgggctgtgatggggcctcaggcggcagcctcaacatggaatgtcgggtgtgcggggacaaggcctcgggcttccactacggggtccacgcgtgcgaggggtgcaagggcttcttccgccggacaatccgcatgaagctcgagtatgagaagtgcgatcggatctgcaagatccagaagaagaaccgcaacaagtgtcagtactgccgcttccagaagtgcctggcactcggcatgtcgcacaacgctatccgctttggacggatgccggacggcgagaagaggaagctggtggcggggctgactgccagcgaggggtgccagcacaacccccagctggccgacctgaaggccttctctaagcacatctacaacgcctacctgaaaaacttcaacatgaccaaaaagaaggcccggagcatcctcaccggcaagtccagccacaacgcaccctttgtcatccacgacatcgagacactgtggcaggcagagaagggcctggtgtggaaacagctggtgaacgggctgccgccctacaacgagatcagtgtgcacgtgttctaccgctgccagtccaccacagtggagacagtccgagagctcaccgagttcgccaagaacatccccaacttcagcagcctcttcctcaatgaccaggtgaccctcctcaagtatggcgtgcacgaggccatctttgccatgctggcctccatcgtcaacaaagacgggctgctggtggccaacggcagtggcttcgtcacccacgagttcttgcgaagtctccgcaagcccttcagtgacatcattgagcccaagttcgagtttgctgtcaagttcaatgcgctggagctcgatgacagtgacctggcgctcttcatcgcggccatcattctgtgtggagaccggccaggcctcatgaatgtgccccaggtagaagccatccaggacaccattctgcgggctctagaattccatctgcaggtcaaccaccctgacagccagtacctcttccccaagctgctgcagaagatggcagacctgcggcagctggtcactgagcatgcccagatgatgcagtggctaaagaagacggagagtgagaccttgctgcaccccctgctccaggaaatctacaaggacatgtactaaggccgcagcccaggcctcccctcaggctctgctgggcccagccacggactgttcagaggaccagccacaggcactggcagtcaagcagctagagcctactcacaacactccagacacgtggcccagactcttcccccaacacccccacccccaccaacccccccattcccccaacccccctcccccaccccgctctccccatggcccgtttcctgtttctcctcagcacctcctgttcttgctgtctccctagcgcccttgctcccccccctttgccttccttctctagcatccccctcctcccagtcctcacatttgtctgattcacagcagacagcccgttggtacgctcaccagcagcctaaaagcagtgggcctgtgctggcccagtcctgcctctcctctctatccccttcaaagggaattc

SEQ ID NO: 4 - protein, 440 aa
MEQPQEETPEAREEEKEEVAMGDGAPELNGGPEHTLPSSSCADLSQNSSPSSLLDQLQMGCDGASGGSLNMECRVCGDKASGFHYGVHACEGCKGFFRRTIRMKLEYEKCDRICKIQKKNRNKCQYCRFQKCLALGMSHNAIRFGRMPDGEKRKLVAGLTASEGCQHNPQLADLKAFSKHIYNAYLKNFNMTKKKARSILTGKSSHNAPFVIHDIETLWQAEKGLVWKQLVNGLPPYNEISVHVFYRCQSTTVETVRELTEFAKNIPNFSSLFLNDQVTLLKYGVHEAIFAMLASIVNKDGLLVANGSGFVTHEFLRSLRKPFSDIIEPKFEFAVKFNALELDDSDLALFIAAIILCGDRPGLMNVPQVEAIQDTILRALEFHLQVNHPDSQYLFPKLLQKMADLRQLVTEHAQMMQWLKKTESETLLHPLLQEIYKDMY

SEQ ID NO: 5 - DNA (cDNA), 23 bp; single-stranded, linear
ggnttycaytayggngtncaycg

SEQ ID NO: 6 - protein, 8 aa
GFHYGVHA